(19) United States
(12) Patent Application Publication
(10) Pub. No.: US 2024/0245099 A1
(43) Pub. Date: Jul. 25, 2024

(54) AEROSOL-GENERATING ARTICLE WITH BIOMARKER SENSOR

(71) Applicant: PHILIP MORRIS PRODUCTS S.A., Neuchatel (CH)

(72) Inventors: Ricardo Cali, Mannheim (DE); Toney Moses Rajan, Mannheim (DE); Alexandra Sereda, Neuchatel (CH)

(21) Appl. No.: 18/561,456

(22) PCT Filed: May 17, 2022
(86) PCT No.: PCT/EP2022/063242

§ 371 Date: Nov. 16, 2023

(30) Foreign Application Priority Data
May 21, 2021 (EP) 21175189.6

(51) Int. Cl. A24D 1/02; A24D 1/20; A24F 40/20; A24F 40/51; G01N 21/78; G01N 33/94

(52) U.S. Cl. CPC A24D 1/025; A24D 1/20; A24F 40/20; A24F 40/51; G01N 33/94; G01N 21/78

(57) ABSTRACT

This invention relates to an aerosol-generating article comprising an aerosol-forming substrate. The aerosol-generating article comprises a mouth end, a distal end, and a biomarker sensor, wherein the biomarker sensor is provided to the aerosol-generating article and at least 1 centimeter distant from the mouth end. This invention also relates to an aerosol-generating device and an aerosol-generating system comprising an aerosol-generating device and an aerosol-generating article. This invention also relates to a method for operating such aerosol-generating system.

The present invention relates to an aerosol-generating article comprising a mouthpiece and a biomarker sensor. The present invention also relates to an aerosol-generating device configured to receive such aerosol-generating article and to a system comprising the aerosol-generating article and the aerosol-generating device. The present invention further relates to a method of using such system.

This disclosure generally relates to aerosol-generating articles that include a biomarker sensor. The aerosol-generating article may be used with an aerosol-generating device. The biomarker sensor can be read out by corresponding control electronics of the aerosol-generating device. In this way feedback may be provided which may be used to adjust the user experience. For example, nicotine delivery may be controlled based on the measured biomarker concentration in the user's saliva. The measured biomarker concentration may preferably be related to nicotine exposure levels of a user.

Biomarkers such as nicotine or its metabolic derivatives such as cotinine or 3-hydroxy-cotinine, may be used for identifying how intensive a user has previously used tobacco products or a nicotine delivering aerosol-generating device. Some metabolic derivatives may be detectable in a user's saliva for more than 40 hours after use.

Thus, by monitoring the biomarker level of a regular user of nicotine delivering products, the nicotine intake can be monitored and may be influenced in a subsequent user experience. Incentives may be provided to keep a user's nicotine intake at a constant or continuously decreasing level.

It would be desirable to provide an aerosol-generating article that is to be used in an aerosol-generating system offering increased functionalities, in particular with respect to monitoring user behaviour.

It would be desirable to provide an aerosol-generating article that is to be used in an aerosol-generating system offering such increased functionality, and which aerosol-generating system is easy to handle. It would be desirable to provide an aerosol-generating system which can be used for an extended time period and which at the same time offers a high hygienic standard.

It would be further desirable to provide an aerosol-generating system comprising an aerosol-generating article that can be manufactured at reduced cost.

According to an embodiment of the present invention there is provided an aerosol-generating article comprising an aerosol-forming substrate. The aerosol-generating article comprises a mouth end and a distal end. The aerosol-generating article comprises a biomarker sensor provided to the aerosol-generating article and at least 1 cm distant from the mouth end.

During a user experience the mouth end of the aerosol-generating article is put into the user's mouth. The biomarker sensor is positioned such that it does not come into direct contact with the user's mouth. In this way the biomarker sensor may be visible throughout the user experience. In particular, the biomarker sensor may be read out by the user or by electronic equipment during the user experience.

The aerosol-generating article may comprise a dedicated mouthpiece at the mouth end of the aerosol-generating article. The mouthpiece is the part of the aerosol-generating article that during the user experience may be placed in the user's mouth. The biomarker sensor may be provided distant from the mouthpiece. The biomarker sensor may be provided upstream from the mouthpiece. By positioning the biomarker sensor distant from the mouthpiece, it may be efficiently ensured that the biomarker does not come into contact with the user's mouth.

The aerosol-generating article may comprise a portion in which the aerosol-forming substrate is provided. The biomarker sensor may be positioned at the portion in which the aerosol-forming substrate is provided. The portion in which the aerosol-forming substrate is provided may not be taken into the user's mouth during a user experience. Accordingly, by positioning the biomarker sensor at the portion in which the aerosol-forming substrate is provided, it may again be efficiently ensured that the biomarker does not come into contact with the user's mouth.

The aerosol-generating article may comprise a perforation. The aerosol-generating article may comprise a perforation line. The biomarker sensor may be provided upstream from the perforation line. Perforation lines are provided in an aerosol-generating article in order to allow ambient air to be introduced into an air flow channel of the aerosol-generating article. In order to fulfil this function, the perforation lines are usually not covered during a user experience. In particular perforation lines are usually not covered by the user's mouth during a user experience. Thus, also in this way it may be efficiently ensured that the biomarker does not come into contact with the user's mouth.

As used herein, an ‘aerosol-generating device’ relates to a device that interacts with an aerosol-forming substrate to generate an aerosol. For example, the aerosol-forming substrate may be part of an aerosol-generating article. The generated aerosol may be an aerosol that is directly inhalable into a user's lungs through the user's mouth. An aerosol-generating device may be a holder. The aerosol-generating device may be an electrically heated aerosol-generating device. The aerosol-generating device may comprise electric circuitry. The aerosol-generating device may comprise a power supply. The aerosol-generating device may comprise a heating chamber. The aerosol-generating device may comprise a heating element. The electric circuitry and the power supply are preferably arranged in the main body of the aerosol-generating device.

As used herein, the term ‘aerosol-generating article’ refers to an article comprising an aerosol-forming substrate that is capable of releasing volatile compounds that can form an aerosol. For example, an aerosol-generating article may be an aerosol-generating article that generates an aerosol that is directly inhalable into a user's lungs through the user's mouth. An aerosol-generating article may be disposable. An aerosol-generating article comprising an aerosol-forming substrate comprising tobacco may be referred to as a tobacco stick.

The aerosol-generating article may be substantially cylindrical in shape. The aerosol-generating article may be substantially elongate. The aerosol-generating article may have a length and a circumference substantially perpendicular to the length. The aerosol-forming substrate may be substantially cylindrical in shape. The aerosol-forming substrate may be substantially elongate. The aerosol-forming substrate may also have a length and a circumference substantially perpendicular to the length.

The aerosol-generating article may have a total length between approximately 30 mm and approximately 100 mm. The aerosol-generating article may have an external diameter between approximately 5 mm and approximately 12 mm. The aerosol-generating article may comprise a filter plug. The filter plug may be located at a downstream end of the aerosol-generating article. The filter plug may be a cellulose acetate filter plug. The filter plug is approximately 7 mm in length in one aspect, but may have a length of between approximately 5 mm to approximately 10 mm.

In one aspect, the aerosol-generating article may have a total length of approximately 45 mm. The aerosol-generating article may have an external diameter of approximately 7.2 mm. Further, the aerosol-forming substrate may have a length of approximately 10 mm. Alternatively, the aerosol-forming substrate may have a length of approximately 12 mm. Further, the diameter of the aerosol-forming substrate may be between approximately 5 mm and approximately 12 mm. The aerosol-generating article may comprise an outer paper wrapper. Further, the aerosol-generating article may comprise a separation between the aerosol-forming substrate and the filter plug. The separation may be approximately 18 mm, but may be in the range of approximately 5 mm to approximately 25 mm.

The heating chamber of the aerosol-generating device may have an elongate shape. The heating chamber of the aerosol-generating device may have a cross-section that corresponds to the cross-section of the aerosol-generating article that is to be used with and inserted into the heating chamber of the aerosol-generating device.

As used herein, the term ‘aerosol-forming substrate’ relates to a substrate capable of releasing volatile compounds that can form an aerosol. Such volatile compounds may be released by heating the aerosol-forming substrate. An aerosol-forming substrate may conveniently be part of an aerosol-generating article.

The aerosol-forming substrate may be a solid or a liquid aerosol-forming substrate. Alternatively, the aerosol-forming substrate may comprise both solid and liquid components. The aerosol-forming substrate may comprise a tobacco-containing material containing volatile tobacco flavour compounds which are released from the substrate upon heating. Alternatively, the aerosol-forming substrate may comprise a non-tobacco material. The aerosol-forming substrate may further comprise an aerosol former that facilitates the formation of a dense and stable aerosol. Examples of suitable aerosol formers are glycerine and propylene glycol.

The aerosol-forming substrate is a substrate capable of releasing volatile compounds that can form an aerosol. The volatile compounds may be released by heating the aerosol-forming substrate.

The aerosol-generating device may comprise electric circuitry. The electric circuitry may comprise a microprocessor, which may be a programmable microprocessor. The microprocessor may be part of a controller. The electric circuitry may comprise further electronic components. The electric circuitry may be configured to regulate a supply of power to the heating element. Power may be supplied to the heating element continuously following activation of the aerosol-generating device or may be supplied intermittently, such as on a puff-by-puff basis. The power may be supplied to the heating element in the form of pulses of electrical current. The electric circuitry may be configured to monitor the electrical resistance of the heating element, and preferably to control the supply of power to the heating element dependent on the electrical resistance of the heating element.

The aerosol-generating device may comprise a power supply, typically a battery, within a main body of the aerosol-generating device. In one aspect, the power supply is a Lithium-ion battery. Alternatively, the power supply may be a Nickel-metal hydride battery, a Nickel cadmium battery, or a Lithium based battery, for example a Lithium-Cobalt, a Lithium-Iron-Phosphate, Lithium Titanate or a Lithium-Polymer battery. As an alternative, the power supply may be another form of charge storage device such as a capacitor. The power supply may require recharging and may have a capacity that enables to store enough energy for one or more usage experiences; for example, the power supply may have sufficient capacity to continuously generate aerosol for a period of around six minutes or for a period of a multiple of six minutes. In another example, the power supply may have sufficient capacity to provide a predetermined number of puffs or discrete activations of the heating element.

The aerosol-generating device may comprise an atomizer. An atomizer is provided to atomize the liquid aerosol-forming substrate to form an aerosol, which can subsequently be inhaled by a user. The atomizer may comprise a heating element, in which case the atomizer will be denoted as a vaporiser. Generally, the atomizer may be configured as any device which is able to atomize the liquid aerosol-forming substrate. For example, the atomizer may comprise a nebulizer or an atomizer nozzle based on the Venturi effect to atomize the liquid aerosol-forming substrate. Thus, the atomization of the liquid aerosol-forming substrate may be realized by a non-thermally aerosolization technique. A mechanically vibrating vaporiser with vibrating elements, vibrating meshes, a piezo-driven nebulizer or surface acoustic wave aerosolization may be used.

Preferably, the atomizer is configured as a vaporiser comprising a heater for heating the supplied amount of liquid aerosol-forming substrate. The heater may be any device suitable for heating the liquid aerosol-forming substrate and vaporize at least a part of the liquid aerosol-forming substrate in order to form an aerosol. The heater may exemplarily be a coil heater, a capillary tube heater, a mesh heater or a metal plate heater. The heater may exemplarily be a resistive heater which receives electrical power and transforms at least part of the received electrical power into heat energy. Alternatively, or in addition, the heater may be a susceptor that is inductively heated by a time varying magnetic field. The heater may comprise only a single heating element or a plurality of heating elements. The temperature of the heating element or elements is preferably controlled by electric circuitry.

As used herein, the terms ‘upstream’, ‘downstream’, ‘proximal’, ‘distal’, ‘front’ and ‘rear’, are used to describe the relative positions of components, or portions of components, of the aerosol-generating device in relation to the direction of the air flow caused by a user inhaling at the mouthpiece of an aerosol-generating device during use thereof.

The biomarker sensor may be provided in any suitable for and shape. The biomarker sensor may be provided a test patch to the outer surface of the aerosol-generating article. The test patch may be adhered with adhesive or any other suitable attachment means to the outer surface of an aerosol-generating article.

The biomarker sensor may be provided in the form of a band that extends around the outer circumference of the aerosol-generating article. Such design may particularly be advantageous for aerosol-generating articles having a cylindrical shape. A biomarker sensor being provided in the form of a band may be firmly fixed to an aerosol generating article using conventional attachment methods. A biomarker sensor that extends around the outer circumference of an aerosol-generating article may be easily observable by a user or by corresponding read-out means throughout the user experience.

The biomarker may be used examine any buccal fluid originating from a user's mouth. Such buccal fluids may mainly include saliva, but may also include for example condensation of a user's breadth. For sake of clarity the term “saliva” is used in this document as an example for any buccal fluid.

The biomarker sensor may employ any suitable biomarker detection technology. Known technologies for detecting biomarker concentrations in a user's saliva include colorimetric methods, gas chromatography (GC), GS-mass spectrometry (GC-MS), high-performance liquid chromatography (HPLC), and radioimmunoassay (RIA). Lateral flow test strips, such as lateral flow point of contact chromographic saliva biomarker detection systems may be used. Such biomarker detection technologies or other suitable technologies can be adapted for use in the present invention.

The biomarker sensor may comprise a substance having a characteristic that changes upon contact with corresponding biomarkers in the user's saliva. The biomarker sensor may contain substances including one or more of nano-particles, dyes, and chemical agents. The biomarker sensor may contain substances, which lead to a change of a physical characteristic when the presence of a specific biomarker in the user's saliva is detected. The substance in the biomarker sensor may be a colorimetric substance configured to change its color upon contact with a biomarker present in the user's saliva.

Chromophore-generating reagents useful for such colorimetric methods may include barbituric acid (BA), 1,3-diethyl-2-thiobarbituric acid (DETBA), and Meldrum's acid (MA). Cotinine equivalent measurements may use cyanide and a chromophore-generating reagent (e.g. BA, MA, DETBA) for determination of pyridine derivatives (specifically nicotine metabolites). BA is known for use with some pyridine derivatives for such colorimetric determinations of cyanide. Colorimetric nicotine-metabolite (such as cotinine) assay may use a pyrazolone as chromophore-generating agent. Such assays may be used for detecting as well as for quantitatively measuring nicotine-metabolite concentration (such as cotinine concentration) in a user's saliva.

The change of color may be visually observed by a user. In this way the user may obtain confirmation about authenticity of the aerosol-generating article. The biomarker sensor may be used for prevention or detection of product counterfeiting. The substance may be provided in a predefined and readily recognizable pattern such as a brand logo, QR-code, a barcode or another visual code, which allows the user to confirm integrity and authenticity of the product.

The biomarker sensor may be a sensor that is responsive to any biomarker present in a user's saliva. Suitable biomarkers include but are not limited to nicotine metabolites or cortisol metabolites.

Any one or more sensors may be configured to detect any one or more nicotine metabolite in a user's saliva. Examples of nicotine metabolites include nicotine glucuronide, nicotine N′-oxide, nicotine isomethonium ion, cotinine methonium ion, cotinine glucuronide, 3-pyridylacetic acid, nicotine-Δ iminium ion, cotinine, cotinine N-oxide, 4-(3-pyridyl)-butanoic acid, 2;-hydroxynicotine, nornicotine, N′-Hydroxymethyl nornicotine, 5′-hydroxycotinine, 7rans-3′-hydroxycotinine, 4-(methylamino)-1-(3-pyridyl)-1-butanone, 4-oxo-4-(3-pyridyl)-butanamide, 4-oxo-4-(3-pyridyl)-N-methylbutanamide, frans-3′-hydroxycotinine glucuronide, 4-(3-pyridyl)-3-butenoic acid, 4-hydroxy-4-(3-pyridyl)-butanoic acid, 4-oxo-4-(3-pyridyl)-butanoic acid, and 5-(-3-pyridyl)-tetrahydro-furan-2-one. At least one sensor may be configured to detect cotinine levels.

Cotinine is a preferred metabolite in part because it has a long plasma-half life and because a high percentage of nicotine is converted to cotinine. For example, cotinine typically has a plasma half-life of from about 11 hours to about 37 hours, compared with about 30 minutes for nicotine. In addition, about 70 percent to about 80 percent of nicotine is converted to cotinine in the liver and delivered to the blood stream. Further, saliva concentrations of cotinine are thought to be proportional to plasma cotinine concentrations.

A biomarker sensor may be configured to quantify an amount of cotinine within a relevant range of concentrations. By way of example, studies have shown that passive exposure to nicotine containing aerosol may result in cotinine concentrations in saliva of below 5 nanograms per milliliter, but heavy passive exposure can results in concentrations in saliva of 10 nanograms per milliliter or greater. Cotinine concentrations in saliva of regular users may range from about 10 nanograms per milliliter to about 100 nanograms per milliliter. Accordingly and preferably, the sensor may be configured to accurately quantify saliva concentrations of cotinine in a range from about 5 nanograms per milliliter to about 200 nanograms per milliliter, such as from about 10 nanograms per milliliter to about 150 nanograms per milliliter. However, it will be appreciated that the range of reliability and sensitivity of the sensor may be tuned to include other concentration ranges as appropriate or desired.

Biomarker sensors may comprise biological substance acting as binding or detecting partners for nicotine metabolites. Such biological substances are generally referred to herein as antibodies to nicotine metabolites. It is known in the prior art how such antibodies may be prepared. Exemplarily it is referred herein to U.S. Pat. No. 5,164,504 (Antibodies for Immunoassays for cotinine derivatives), U.S. Patent application No. 2011/305715 (Antibodies to 3-hydroxycotinine) and U.S. Pat. No. 7,517,699 (Lateral flow Cotinine immunoassay), which are all incorporated herein by reference in its entirety to the extent that they do not conflict with the present disclosure.

Sensor technology that can be employed to achieve or approach the above mentioned results is described by, for example, Francesco Riccia, b, Gianluca Adornettoa, Giuseppe Palleschia, ELECTROCHEMICAL SCIENCE AND TECHNOLOGY State of the Art and Future Perspectives On the occasion of the International Year of Chemistry (2011); Electrochimica Acta; Volume 84, 1 Dec. 2012, Pages 74-83, which is incorporated herein by reference in its entirety to the extent that it does not conflict with the present disclosure. Further description of suitable sensor technologies can be found in Ashlesha Bhide, et al., “Next-Generation Continuous Metabolite Sensing toward Emerging Sensor Needs”, ACS Omega 2021, 6, 6031-6040, in Shikha Sharma et al., “Antibodies and antibody-derived analytical biosensors”, Essays in Biochemistry (2016) 60 9-18, and in Nikhil Bhalla et al., Introduction to biosensors Essays in Biochemistry (2016) 60 1-8, which are all incorporated herein by reference in its entirety to the extent that they do not conflict with the present disclosure.

The aerosol-generating article may comprise a plurality of biomarker sensors. The biomarker sensors may all be responsive to the same biomarker. The sensors may be responsive to different biomarkers. By using a plurality of biomarker sensors a biomarker signature or a molecular signature may be obtained.

In order to provide saliva to the biomarker sensor, a user may be required to lick using the tongue the cigarette paper or the biomarker band. This licking on the biomarker band or the cigarette paper allows the user saliva to directly absorb and make the saliva available for the chemical reaction in the biomarker sensor. An optically visible change in color of the biomarker sensor may be perceivable shortly after application of the user's saliva.

The user's saliva may also be transported to the biomarker sensor during normal use of the aerosol-generating article. For this purpose the aerosol-generating article may comprise one or more capillary channels extending between the biomarker sensor and the mouth end of the aerosol-generating article.

The mouth end of the aerosol-generating article is to be taken into a user's mouth during the user experience. Accordingly a capillary channel extending from the mouth end of the aerosol-generating article may be advantageously used to transfer saliva from the user's mouth towards the biomarker sensor of the aerosol-generating article.

In case a plurality of biomarker sensors are used, a plurality of capillary channels may be employed. Each of the capillary channels may be operably coupled to one of the biomarker sensors.

The aerosol-generating article may be elongate and may define a longitudinal axis. The one or more capillary channels may extend in a direction substantially parallel to the longitudinal axis of the aerosol-generating article.

A capillary channel may be formed as a hollow tube. A capillary channel may be formed from polymeric or composite material.

The diameter of the capillary channel may range between 0.001 and 1.0 millimeters. The diameter of the capillary channel may range between 0.01 and 0.5 millimeters. The diameter of the capillary channel may range between 0.01 and 0.1 millimeters. The diameter of the capillary channel may depend on the fluid material that is to be transported by capillary action. Low viscosity materials generally require smaller diameter capillary tubes in order to achieve sufficiently fast transport.

The capillary channel may be a hollow channel having a non-circular inner cross-section. The diameter of such non-circular capillary channel is to be understood as the cross-sectional dimension having the largest extension.

The aerosol-generating article may comprise three, four or five capillary channels. The capillary channels may have identical dimensions. The capillary channels may differ in length. The capillary channels may differ in diameter. The capillary channels may differ in length and diameter.

The aerosol-generating article may comprise a mouthpiece. The mouthpiece may comprise a mouthpiece core that is enclosed by a wrapper, a tipping paper or by both a wrapper and a tipping paper. The wrapper and the tipping paper may be formed from any suitable material or combination of materials. The wrapper or the tipping paper may be formed from paper, laminated paper, or cellulosic material. The wrapper or the tipping paper may be formed from cigarette paper.

A mouthpiece core may be formed from material that is usually used in manufacture of cigarette filters. The mouthpiece core may comprise a filter. The filter may be formed from one or more suitable filtration materials. Many such filtration materials are known in the art. In one embodiment, the mouthpiece core may comprise a filter formed from cellulose acetate tow. The mouthpiece core may comprise a hollow acetate tube.

The mouthpiece may have an external diameter that is approximately equal to the external diameter of the aerosol-generating article.

The mouthpiece may have an external diameter of a diameter of between approximately 5 millimetres and approximately 10 millimetres.

The mouthpiece may have a length of between approximately 5 millimetres and approximately 20 millimetres.

The mouthpiece may comprise a capillary channel. The mouthpiece core and the capillary channel may be circumscribed by a wrapper.

The capillary channel may extend in a direction generally parallel to the longitudinal direction of the mouthpiece, and may be positioned, in radial direction, between the mouthpiece core and the wrapper.

The aerosol-generating article may comprise further components upstream from the mouthpiece. Such further components may include a portion with sensorial medium. The biomarker sensor may be provided at any portion of the aerosol-generating article except at the mouthpiece. The capillary channel may extend from the mouth end of the aerosol-generating article to the location where the biomarker sensor is positioned.

The biomarker sensor may be provided at the sensorial medium portion of the aerosol-generating article. The capillary channel may extend from the mouth end of the aerosol-generating article to the biomarker sensor positioned in the sensorial medium portion.

By providing the biomarker sensor in a portion that is not taken into the mouth of the user, it may be ensured that the reaction of the biomarker sensor upon exposure to the user's saliva can be visually inspected by the user throughout the user experience.

The biomarker sensor may be provided at the outer surface of the aerosol-generating article. In this way it can be ensured that the biomarker sensor can be visually inspected by the user or by a corresponding sensor arrangement throughout the user experience.

The biomarker sensor may also be covered by one or more layers of transparent, semi-transparent or perforated material. Such materials may be useful to protect the biomarker sensor during handling of the aerosol-generating article.

In order to still allow the user's saliva to arrive at the biomarker sensor, the outer wrapping material of aerosol-generating article may be configured as a paper microfluidic device. Such paper microfluidic device may include the use of hydrophilic cellulose fibers provided between hydrophobic barriers. Such paper microfluidic devices may transport fluids by capillary force and are well known in the art. Paper microfluidic devices may be prepared by wax printing, inkjet printing, photolithography, flexographic printing, plasma treatment, laser treatment, wet etching, screen-printing, or wax screen-printing. Manufacture of such paper microfluidic devices may also comprise a plurality of paper layers that are stacked to form a 3D arrangement of capillary channels.

The thickness of the layers of transparent, semi-transparent or perforated material needs to be configured such that the biomarker sensor can still be visually inspected by the user or by a corresponding sensor arrangement. The overall thickness of these layers may be below 1.0 millimeters. The overall thickness of these layers may be below 0.1 millimeters.

The present invention also relates to an aerosol-generating device comprising a cavity for receiving an aerosol-generating article as described above. The cavity may form the heating chamber of the aerosol-generating device. The aerosol-generating device may comprise a sensor arrangement configured to detect a change of the characteristic of the biomarker sensor. The sensor arrangement of the aerosol-generating device may comprise an optical sensor configured to detect a colorimetric change of the biomarker sensor.

The sensor arrangement of the aerosol-generating device may be positioned such that it can read out the biomarker sensor in use when the aerosol-generating article is inserted into the cavity of the aerosol-generating device.

If the biomarker sensor is provided in the form of a test patch that does not extend over the full circumference of the aerosol-generating article, it has to be ensured that the aerosol-generating article is inserted into the cavity of the aerosol-generating device in the correct rotational orientation, so as to make sure that the sensor arrangement and the biomarker sensor are located in a read-out position next to each other. Alternatively, the sensor arrangement may be provided such that it is able to monitor the complete circumference of the aerosol-generating article. In such case, it is only required that the aerosol-generating article is inserted in such way that the axial position of the biomarker sensor corresponds to the axial position of the sensor arrangement. In this case the biomarker sensor can be read out independent of the rotational orientation of the aerosol-generating article.

If the biomarker sensor is provided in the form of a band that does extend over the full circumference of the aerosol-generating article, the biomarker sensor can also be read out independent of the rotational orientation of the aerosol-generating article. This may facilitate handling of the aerosol-generating device.

Suitable sensor arrangements for reading out the biomarker sensor are well known to the skilled person and need not to be described in more detail herein. In particular sensor arrangements for reading out colorimetric data of a biomarker sensor are known in this regard. Optical sensor arrangements may include a camera for determining a colour change of a biomarker sensor. Such optical sensor arrangements may be able to determine the colour change of the biomarker sensor and may therefore be able to determine whether a given biomarker is present in the user's saliva. It is also possible to determine with such optical sensor arrangements to quantify the colour change of the biomarker sensor and to thereby determine a concentration level of the biomarker in the user's saliva.

The aerosol-generating device may comprise a control unit that is operably coupled to the sensor arrangement. The control unit may be configured to use the data provided from the sensor arrangement for controlling operation of the aerosol-generating device. The control unit may be configured to control operation of the aerosol-generating device based on the biomarker concentration levels obtained by the biomarker sensor of the aerosol-generating article.

By evaluating the biomarker data provided from the sensor arrangement control of the aerosol-generating device can be adapted to the determined biomarker data or the determined biomarker signature. In this way the individual user experience can be enhanced. For example the nicotine delivery can be adjusted based on the biomarker data. Should the biomarker data show conspicuous biomarker levels, operation of the aerosol-generating device may be limited or prevented.

The present invention also relates to an aerosol-generating system comprising an aerosol-generating article and an aerosol-generating device as described above.

The present invention also relates to a method of operating an aerosol-generating system. The method may include the steps of inserting an aerosol-generating article as described above into an aerosol-generating device. The aerosol-generating device comprises a control unit that is operably coupled to a sensor arrangement for reading out the biomarker sensor of the aerosol-generating article. Operation of the aerosol-generating device is controlled based on the biomarker concentration levels determined by the biomarker sensor of the aerosol-generating article.

Below, there is provided a non-exhaustive list of non-limiting examples. Any one or more of the features of these examples may be combined with any one or more features of another example, embodiment, or aspect described herein.

Example 1: Aerosol-generating article comprising an aerosol-forming substrate, the aerosol-generating article comprising a mouth end and a distal end, and a biomarker sensor, wherein the biomarker sensor is provided to the aerosol-generating article and at least 1 centimeter distant from the mouth end.

Example 2: Aerosol-generating article according to example 1, further comprising a mouthpiece at the mouth end and wherein the biomarker sensor is provided distant from the mouthpiece.

Example 3: Aerosol-generating article according to any preceding example, further comprising a portion in which the aerosol-forming substrate is provided and wherein the biomarker sensor is positioned at the portion in which the aerosol-forming substrate is provided.

Example 4: Aerosol-generating article according to any preceding example, further comprising a perforation and wherein the biomarker sensor is provided upstream from the perforation.

Example 5: Aerosol-generating article according to any preceding example wherein the biomarker sensor is a test patch provided to the outer surface of the aerosol-generating article.

Example 6: Aerosol-generating article according to any preceding example, wherein the aerosol generating article has a cylindrical shape and the biomarker sensor is provided in the form of a band around the outer circumference of the aerosol-generating article.

Example 7: Aerosol-generating article according to any preceding example, wherein the biomarker sensor comprises a substance having a characteristic that changes upon contact with corresponding biomarkers in the user's saliva.

Example 8: Aerosol-generating article according to any preceding example, wherein the substrate in the nicotine metabolite sensor comprises one or more of dyes, and chemical agents.

Example 9: Aerosol-generating article according to any preceding example, wherein the substrate in the biomarker sensor is a colorimetric substance configured to change its color upon contact with nicotine or nicotine metabolites present in the user's saliva.

Example 10: Aerosol-generating article according to any preceding example, comprising one or more capillary channels extending between the biomarker sensor and the mouth end of the aerosol-generating article.

Example 11: Aerosol-generating article according to any preceding example, wherein the one or more capillary channels may be polymer or composite tubes having an inner diameter of below 0.1 millimeter and preferably below 0.01 millimeter.

Example 12: Aerosol-generating article according to any preceding example, comprising a mouthpiece and a capillary channel extending along the length of the mouthpiece.

Example 13: Aerosol-generating article according to any preceding example, wherein the mouthpiece comprises a mouthpiece core material and a capillary channel, wherein the mouthpiece core material and the capillary channel are enclosed by a wrapper.

Example 14: Aerosol-generating article according to any preceding example, wherein the capillary channel is positioned radially between the mouthpiece core material and the wrapper.

Example 15: Aerosol-generating article according to any preceding example, wherein the biomarker sensor is positioned such that it can be visually inspected by the user.

Example 16: Aerosol-generating article according to any preceding example, wherein the biomarker sensor is positioned at the outer surface of the aerosol-generating article.

Example 17: Aerosol-generating article according to any preceding example, wherein the biomarker sensor is covered by one or more layers of transparent, semi-transparent or perforated material.

Example 18: Aerosol-generating article according to any preceding example, wherein a plurality of biomarker sensors is used, the biomarker sensors being responsive to the same or to different biomarkers.

Example 19: Aerosol-generating device comprising a cavity for receiving an aerosol-generating article according to any preceding example, wherein the aerosol-generating device comprises a sensor configured to detect a change of the characteristic of the biomarker sensor.

Example 20: Aerosol-generating device according to example 19, wherein the aerosol-generating device comprises an optical sensor configured to detect a colorimetric change of the biomarker sensor.

Example 21: Aerosol-generating system comprising an aerosol-generating article according to any of examples 1 to 18 and an aerosol-generating device according to any of examples 19 and 20.

Example 22: Aerosol-generating system according to example 21, wherein the aerosol-generating device comprises control unit that is operably coupled to the sensor and which controls operation of the aerosol-generating device based on the biomarker concentration levels obtained by the biomarker sensor of the aerosol-generating article.

Example 23: Method of operating an aerosol-generating system according to examples 21 or 22, wherein the aerosol-generating device comprises a control unit that is operably coupled to the sensor and wherein operation of the aerosol-generating device is controlled based on the biomarker concentration levels obtained by the biomarker sensor of the aerosol-generating article.

Features described in relation to one embodiment may equally be applied to other embodiments of the invention.

The invention will be further described, by way of example only, with reference to the accompanying drawings in which:

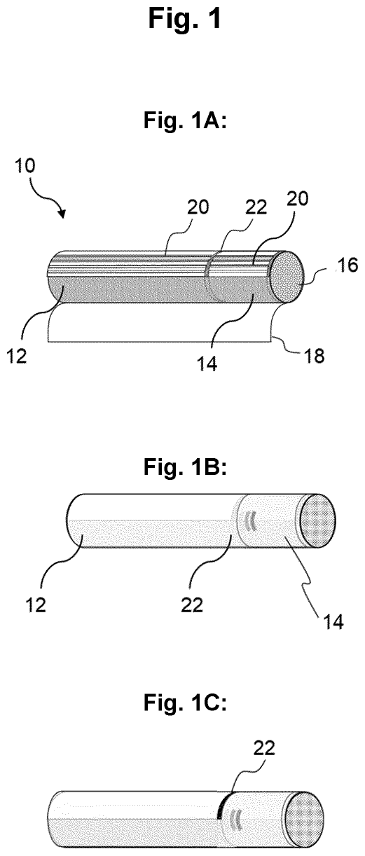

FIG. 1 shows an aerosol-generating article with capillary channels;

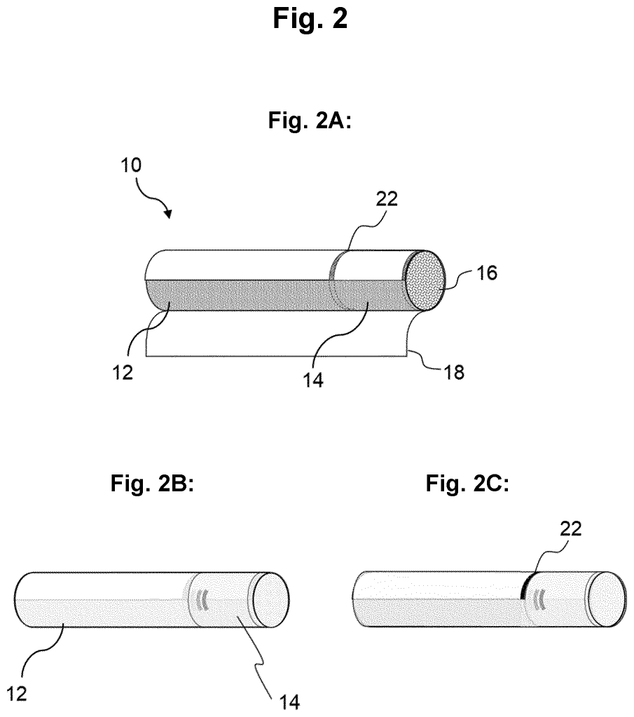

FIG. 2 shows an aerosol-generating article with a biomarker sensor;

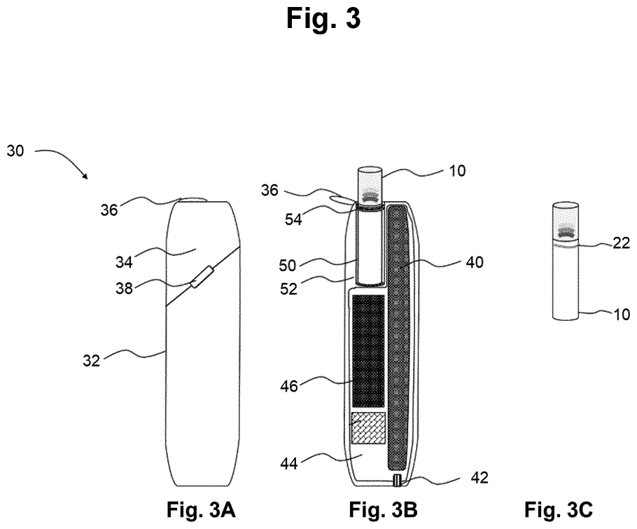

FIG. 3 shows an aerosol-generating device of the present invention;

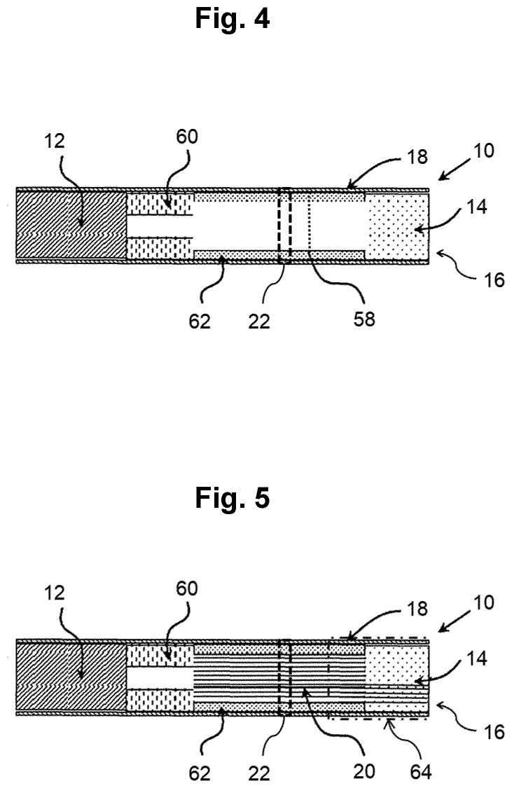

FIG. 4 shows an aerosol-generating article with a perforation;

FIG. 5 shows an aerosol-generating article with a filter tipping paper.

FIG. 1 shows an aerosol-generating article 10 in an embodiment of the present invention. As depicted in FIG. 1A the cylindrical aerosol-generating article 10 comprises a sensorial medium portion 12 and a mouthpiece portion 14. The mouthpiece portion 14 is provided at the mouth end 16 of the aerosol-generating article 10. Both portions are wrapped with and connected to each other by a wrapper 18.

Along the full length of the aerosol-generating article 10 capillary channels 20 are provided. These capillary channels 20 are hollow polymeric tubes having an inner diameter of 0.01 millimeter. The capillary channels are provided over the circumference of the sensorial media portion 12 and the mouthpiece portion 14 and are sandwiched between these portions 12, 14 and the wrapper 16. Wrapper 18 is made from conventional cigarette paper.

Orthogonal to the capillary channels 20 a biomarker sensor 22 is provided. In this case, the biomarker sensor 22 comprises pyrazolone, which is useful for determining nicotine and cotinine concentrations in a user's saliva.

The biomarker sensor 22 is provided in the form of a band that extends over the full circumference of the aerosol-generating article 10. The biomarker sensor 22 is operably coupled to the capillary channels 20. Fluid, in particular a user's saliva, may enter into the capillary channels 20 from the mouth end 16 when the aerosol-generating article 10 is placed in a user's mouth during a user experience.

FIG. 1B shows a fully wrapped aerosol-generating article 10. The wrapper is provided with different colors to identify the sensorial media portion 12 and the mouthpiece portion 14 of the aerosol-generating article 10. As depicted in FIG. 1B the biomarker sensor 22 is provided at the sensorial medium portion 12 of the aerosol-generating article 10. As long as no nicotine or cotinine is detected at the biomarker sensor 22, the biomarker sensor 22 is colorless and not recognizable on the aerosol-generating article 10.

During use of the aerosol-generating article 10 the mouthpiece portion is placed in a user's mouth. The physical contact of the aerosol-generating article 10 with a user's mouth allows saliva to enter into the capillary channels 20. The saliva is drawn by capillary action towards and onto the biomarker sensor 22. If the user's saliva contains nicotine or cotidine, these biomarkers react with the pyrazolone of the biomarker sensor and change the color of the biomarkers sensor 22 as schematically indicated in FIG. 1C. This change of color can be visually determined by the user and may also be detected by a corresponding sensor arrangement of an aerosol-generating device.

FIG. 2 shows an alternative arrangement of an aerosol-generating article 10 of the present invention. The aerosol-generating article 10 largely corresponds to the aerosol-generating article 10 of FIG. 1. The aerosol-generating article 10 of FIG. 2 does not comprise any capillary channels. Thus, in order to allow user's saliva to come into contact with the biomarker sensor 22, the user may lick the biomarker sensor 22 or the portion of the cigarette paper wrapper 18 comprising the biomarker sensor 22 directly with the user's tongue.

Again, if the user's saliva contains nicotine or cotidine, a change of color will occur that can be visually determined by the user and may also be detected by a corresponding sensor arrangement of an aerosol-generating device.

In FIG. 3 an aerosol-generating device 30 for use with an aerosol-generating article 10 is depicted. The aerosol-generating device 30 and the aerosol-generating article 10 together form an aerosol generating system.

As depicted in FIG. 3A, the aerosol-generating device 30 comprises a housing 32 with a removable cap 34. The removable cap 34 comprises a cover 36 which covers an opening in the removable cap 34. The aerosol-generating device 30 has a button 38 via which the aerosol-generating device 30 may be switched on and off.

In the cross-sectional view of FIG. 3B the internal components of the aerosol-generating device 30 are depicted. The aerosol-generating device 30 comprises a battery 40, and a charging port 42, and a printed circuit board 44 with a control unit 46.

The aerosol-generating device defines a cavity 50 that is accessible via the opening in the removable cap 34. The cavity 50 comprises an electric heater 52 and acts as a heating chamber for heating the sensorial medium portion of an aerosol-generating article.

A sensor arrangement is provided within the cavity 50. The sensor arrangement comprises an optical sensor 54. The optical sensor 54 is ring shaped and extends around the inner surface of the cavity 50. The optical sensor 54 is located in direct line of sight of the biomarker sensor 22 of an inserted aerosol-generating article 10.

FIG. 3C shows an aerosol-generating article 10 according to FIG. 1 or 2 comprising a band shaped biomarker sensor 22. As can be seen form a comparison of FIGS. 3B and 3C the vertical position of the biomarker sensor 22 corresponds to the vertical position of the optical sensor 54, when the aerosol-generating article 10 is fully inserted into the cavity of the aerosol-generating device 30.

The control unit of the aerosol-generating device 30 is coupled with the optical sensor 54. The optical sensor 54 is configured to monitor the biomarker sensor 22. When the biomarker sensor 22 changes its color due to the presence of nicotine or cotinine in a user's saliva, the optical sensor 54 may generate a corresponding signal that is received by the control unit 46. Based on the data received from the optical sensor 54, the control unit 46 may adjust operation of the aerosol-generating device 30.

FIG. 4 shows a further embodiment of an aerosol-generating article 10 of the present invention. The aerosol-generating article 10 comprises a sensorial medium portion 12 and a mouthpiece portion 14 that are connected by a wrapper 18. Between the sensorial medium portion 12 and a mouthpiece portion 14 there are provided additional components 60, 62. These additional components 60, 62 are hollow acetate tubes with different diameters, that are used to assist in aerosol formation within the air flow path defined in the aerosol-generating article 10. A perforation line 58 comprising a plurality of perforations is provided in the area of component 62, which is the larger diameter hollow acetate tube. A biomarker sensor 22 is provided upstream from the perforation line 58. Since perforation line 58 is not to be covered by a user's mouth during the user experience, the biomarker sensor 22, which is located even further away from the mouth end 16 of the aerosol-generating article 10, will equally not be in direct contact with the user's mouth during the user experience. In order to transfer a user's saliva to the biomarker sensor 22, the biomarker sensor 22 may be licked by the user in accordance with the embodiment described with FIG. 2, or capillary tubes may be provided at the aerosol-generating article 10 in accordance with the embodiment described with FIG. 1.

FIG. 5 shows also an embodiment of an aerosol-generating article 10 of the present invention, which is similar to the aerosol-generating article 10 of FIG. 4. In this embodiment no perforation line is provided. However, the portion that is potentially in contact with a user's mouth is additionally wrapped with a tipping paper 64 having a different color than wrapper 18. The biomarker sensor 22 is provided upstream from the region covered by the tipping paper 64. Thus, again the biomarker sensor 22 is provided in an area of the aerosol-generating article 10, which will not be in direct contact with the user's mouth during the user experience. In order to transfer a user's saliva to the biomarker sensor 22 capillary tubes 20 are provided that extend from the mouth end 16 of the aerosol-generating article 10 and which are operably coupled to the biomarker sensor 22. For better visibility only a part of the capillary tubes 20 are indicated in FIG. 5 to extend from the mouth end 16 of the aerosol-generating article 10. Typically, all capillary tubes 20 will be configured to extend from the mouth end 16 of the aerosol-generating article 10.

1. Aerosol-generating article comprising an aerosol-forming substrate,
the aerosol-generating article comprising a mouth end and a distal end, and a biomarker sensor,
wherein the biomarker sensor is provided to the aerosol-generating article and at least 1 centimeter distant from the mouth end.

2. Aerosol-generating article according to claim 1, further comprising a mouthpiece at the mouth end and wherein the biomarker sensor is provided distant from the mouthpiece.

3. Aerosol-generating article according to claim 1, further comprising a portion in which the aerosol-forming substrate is provided and wherein the biomarker sensor is positioned at the portion in which the aerosol-forming substrate is provided.

4. Aerosol-generating article according to claim 1, further comprising a perforation and wherein the biomarker sensor is provided upstream from the perforation.

5. Aerosol-generating article according to claim 1, wherein the aerosol-generating article has a cylindrical shape and the biomarker sensor is provided in the form of a band around an outer circumference of the aerosol-generating article.

6. Aerosol-generating article according to claim 1, wherein the biomarker sensor comprises a substance having a characteristic that changes upon contact with corresponding biomarkers in user's saliva.

7. Aerosol-generating article according to claim 1, wherein the substrate in the biomarker sensor is a colorimetric substance configured to change its color upon contact with nicotine or nicotine metabolites present in user's saliva.

8. Aerosol-generating article according to claim 1, wherein the biomarker sensor is positioned such that it can be visually inspected by a user.

9. Aerosol-generating article according to claim 1, wherein the biomarker sensor is positioned at an outer surface of the aerosol-generating article.

10. Aerosol-generating article according to claim 1, wherein the biomarker sensor is covered by one or more layers of transparent, semi-transparent or perforated material.

11. Aerosol-generating device comprising a cavity for receiving an aerosol-generating article according to claim 1, wherein the aerosol-generating device comprises a sensor configured to detect a change of a characteristic of the biomarker sensor of the aerosol-generating article.

12. Aerosol-generating device according to claim 11, wherein the aerosol-generating device comprises an optical sensor configured to detect a colorimetric change of the biomarker sensor of the aerosol-generating article.

13. Aerosol-generating system comprising an aerosol-generating article according to claim 1 and an aerosol-generating device comprising a cavity for receiving the aerosol-generating article, wherein the aerosol-generating device comprises a sensor configured to detect a change of a characteristic of the biomarker sensor of the aerosol-generating article.

14. Aerosol-generating system according to claim 13, wherein the aerosol-generating device comprises control unit that is operably coupled to the sensor and which controls operation of the aerosol-generating device based on a biomarker concentration levels obtained by the biomarker sensor of the aerosol-generating article.

15. Method of operating an aerosol-generating system according to claim 13, wherein the aerosol-generating device comprises a control unit that is operably coupled to the sensor and wherein operation of the aerosol-generating device is controlled based on a biomarker concentration levels obtained by the biomarker sensor of the aerosol-generating article.
